(19) United States
(12) Patent Application Publication
(10) Pub. No.: US 2006/0247204 A1
(43) Pub. Date: Nov. 2, 2006

(54) New therapeutic use of chondroitin sulphate

(76) Inventors: Francisco Javier Vila Pahi, Barcelona (ES); Josep Verges Milano, Corbera De Llobregat (ES); Montserrat Perez Lopez, Barcelona (ES)

Correspondence Address: SUGHRUE MION, PLLC, 2100 PENNSYLVANIA AVENUE, N.W., SUITE 800, WASHINGTON, DC, 20037, US

(21) Appl. No.: 10/567,061

(22) PCT Filed: Jul. 16, 2004
(86) PCT No.: PCT/EP04/07902

§ 371 Date: Feb. 3, 2006

(30) Foreign Application Priority Data
Aug. 6, 2003 (ES) P 200301899

(51) Int. Cl. A61K 31/737

(52) U.S. Cl. 514/54

(57) ABSTRACT

The present invention relates to the use of an alkaline or alkaline earth metal chondroitin sulphate, which comes from an enzymatic hydrolysis of animal cartilage, for the preparation of a medicament for the treatment or prevention of psoriasis with skin affection in a mammal. Preferably the sodium chondroitin sulphate has an average molecular weight between 10,000 and 20,000 daltons, and is administered orally.

### TECHNICAL FIELD OF THE INVENTION

The present invention relates to the use of alkaline or alkaline earth metal chondroitin sulphate, which comes from an enzymatic hydrolysis of animal cartilage, for the treatment or prevention of psoriasis with skin affection.

### BACKGROUND OF THE INVENTION

Psoriasis with skin affection has great clinical polymorphism (E. Christophers, Clin. Exp. Dermatol. 26(4), 314-320 (2001)). Clinically, the skin lesion manifests itself in the form of an erythematous plaque with clear edges, covered by thick, whitish scales, with a waxen appearance, which are preferably distributed over large areas. It is characterized in that it has a proliferation of epidermal keratinocytes and a lack of maturity of these cells in the formation of normal keratin. It affects 2% of the white population at some time in their life and there are important differences between different ethnic groups. In Europe, the prevalence varies between 1.5% in Croatia, and 4.8% in Norway. The evolution of the disease is unpredictable and it has been demonstrated that it intensely affects the patient's quality of life (S. R. Rapp et al., Br. J. Dermatol. 145, 610-616 (2001)).

The exact cause of psoriasis with skin affection is unknown. It seems that exogenous factors act, on top of a certain genetic predisposition, which makes the disease appear or reappear. Amongst these we have: traumatisms; streptococcal infections; endocrine disturbances such as puberty, menopause, postpartum, estrogenic treatment; metabolic factors such as hypocalcemia or dialysis; psychogenic factors such as stress or alcoholism and drugs (systemic corticosteroids, aspirin, penicillin, nystatin, non-steroidal anti-inflammatory drugs or NSAIDs, etc.). Furthermore, the interruption of corticosteroids can cause a new break-out of psoriasis.

Psoriasis with skin affection is a chronic disease that has no definitive treatment. Current medical treatment depends on the type of lesion, location and patient's age (R. Gniadecki et al., Acta Derm. Venereol. 82, 401-410 (2002)). We will highlight the following therapeutic possibilities:

Topical corticoids have anti-inflammatory, antiproliferative, immunosupressive and vasoconstrictive actions. The cutaneous secondary effects are stretch marks, cutaneous atrophy, ecchymosis, perioral dermatitis, rosacea, acne, allergic contact dermatitis, slow wound healing, hypertrichosis, glaucoma, folliculitis, millaria and hypopigmentation, amongst others. Systemic corticoids are permitted in cases of serious psoriasis.

Dithranol (anthralin) is effective in the treatment of psoriasis but should be used with precaution as it has two important drawbacks. The first is that it stains of purple clothes and the skin surrounding the psoriatic lesions. The second is that it irritates the unaffected skin and, if used at an excessive potency, will cause pain, erythema and the formation of vesicles.

Coal-tar preparations are currently used to a much less degree. Their main problem lies in the fact that they have a strong smell and are also very messy.

Calcipotriol is a vitamin D analogue (C. M. Popescu et al., Arch. Dermatol. 136(12), 1547-1549 (2000)), which has the advantages, in relation to other topical preparations, of its ease of use and lack of effect on dermal collagen. Nevertheless, even though calcipotriol has anti-psoriatic properties, it does not completely whiten the lesions and, in some patients, provides no benefits.

Phototherapy is performed by irradiating the skin with mid-range ultraviolet radiation (UVB). The drawback of this treatment is that it requires times and the patients need to go to specialized centres.

Photochemotherapy (PUVA) (topical or systemic) consists of the topical or systemic use of a photosensitizer (8-MOP), with subsequent irradiation of the skin with UVA. This treatment is effective, but should be reserved for psoriasis in highly extended plaques or those that quickly relapse after topical treatments.

Acitretin is a retinoid that shows beneficial effects in psoriasis. Secondary effects include cheilitis and xeroderma and its prolonged use can cause hyperostosis and metaphysis closure in children and ligament calcification in adults. Hypertriglyceridemia is frequent.

Methotrexate is an effective medicine to treat psoriasis (B. Kumar et al., nt. J. Dermatol. 41(7), 444-448 (2002)). Nevertheless, a proportion of patients develop a sensation of nausea and lethargy 48 hours after the dose is administered. Main secondary effects are gastrointestinal ulceration and bone marrow depression, amongst others. Furthermore, this compound is hepatoxic and can cause hepatic fibrosis, particularly after prolonged treatments.

Cyclosporin is the first medicine used in psoriasis based on knowledge of pathogenesis of the disease rather than empirical knowledge (W. P. Gulliver, Cutis 66(5), 365-369 (2000)). Although it is effective at curing psoriasis, the most serious secondary effects include hypertension and nephrotoxicity and, for this reason, the renal function must, particularly, be carefully monitored.

Glycosaminoglycans are polymeric biomolecules of high molecular weight, which are basically found in living organisms where they perform different physiological functions.

Chondroitin sulphate is a sulphated glycosaminoglycan with a polymeric structure characterized in that it has a repeated disaccharide unit, formed by N-acetylgalactosamine and glucuronic acid. Most of the N-acetylgalactosamine residues are sulphated.

The chondroitin sulphate which comes from cartilaginous tissue is mainly found in two isomeric forms which differ in the position of the sulphate group present in the N-acetylgalactosamine residue. Chondroitin 4-sulphate (chondroitin sulphate A) which mainly contains the disaccharide unit [->4)-O-(β-D-glucopyranosyluronic acid)-(1->3)-O-(2-acetamido-2-deoxy-β-D-galactopyranosyl-4-sulphate)-(1->] and chondroitin 6-sulphate (chondroitin sulphate C), which contains the disaccharide unit [->4)-O-(β-D-glucopyranosyluronic acid)-(1->3)-O-(2-acetamido-2-deoxy-β-D-galactopyranosyl-6-sulphate)-(1->].

When the specification of the present invention speaks of chondroitin sulphate, it refers to chondroitin 4-sulphate, chondroitin 6-sulphate, chondroitin sulphate where some of the repeated disaccharide units are non-sulphated, chondroitin sulphate where some of the repeated disaccharide units are polysulphated, chondroitin sulphate where the repeated disaccharide unit is polysulphated, and mixtures thereof.

The use of chondroitin sulphate has been disclosed to treat various illnesses, e.g. in the treatment of cardiovascular diseases (M. Morrison et al. U.S. Pat. No. 3,895,106), however, the most widespread use of chondroitin sulphate is in the treatment of osteoarthritis (arthrosis), which is characterized by a joint dysfunction, with reduction and loss of cartilage (M. G. Lequesne, Rev. Rhum. Eng. Ed., 61, 69-73 (1994), P. Morreale et al., J. Rheumatology 23, 1385-1391 (1996) and G. Verbruggen et al., Osteoarthritis Cart., 6 (Supplement A), 37-38 (1998)).

The chondroitin sulphate which is commonly used in therapy is in the form of sodium salt.

O. Olsen et al. (WO 01/83707) disclose a process to produce antiangiogenic, anti-inflammatory, lixozymic and/or anti-collagenolytic and/or collagen and/or chondroitin sulphate fractions from chondrocytes cultured in vitro. The patent application claims a process for the treatment or prevention of psoriasis using chondroitin sulphate obtainable from chondrocytes cultured in vitro, but said patent application does not disclose how to extract said chondroitin sulphate and therefore states nothing regarding its characteristics and activity.

It is known that the structure of a chondroitin sulphate varies depending on the animal species, the tissue it comes from and the process to obtain it.

It is also known (M. Morrison et al. U.S. Pat. No. 3,895,106) that the differences in the activity of different chondroitin sulphates may be both due to the differences in its preparation process and the use of the different starting material.

Indeed, O. Olsen et al., (WO 01/83707) consider that the chondroitin sulphate obtainable from cultured chondrocytes is a new product, since the claim is of the “product by process” type (new product which can de defined and claimed according to its preparation process).

Until now, the use of chondroitin sulphate, which comes from an enzymatic hydrolysis of animal cartilage, has not been disclosed in the treatment of psoriasis with skin affection.

From this, we can gather that providing a drug which is useful in the treatment of psoriasis with skin affection, without the drawbacks or secondary effects of the current therapies, is still an important problem of the therapeutics.

### DISCLOSURE OF THE INVENTION

Unexpectedly, it has been observed that chondroitin sulphate, which comes from an enzymatic hydrolysis of animal cartilage, is useful in the treatment of psoriasis with skin affection. The present invention therefore relates to the use of alkaline or alkaline earth metal chondroitin sulphate which comes from an enzymatic hydrolysis of animal cartilage, for the preparation of a medicament for the treatment or prevention of psoriasis with skin affection in a mammal.

In a preferred embodiment the animal cartilage is bovine, porcine or cartilaginous fish cartilage.

In a more preferred embodiment the alkaline metal chondroitin sulphate is sodium chondroitin sulphate.

More preferred is the sodium chondroitin sulphate which has an average molecular weight between 10,000 and 40,000 daltons.

Likewise, more preferred is the sodium chondroitin sulphate which has an average molecular weight between 10,000 and 20,000 daltons.

In an even more preferred embodiment, the sodium chondroitin sulphate which has an average molecular weight between 10,000 and 20,000 daltons, has a sulphur content between 5% and 7% weight/weight, on anhydrous base.

In an especially preferred embodiment, the medicament is adapted for oral administration, which comprises 200 to 3,000 mg of sodium chondroitin sulphate daily.

Likewise, in an especially preferred embodiment, the medicament is adapted for topical administration.

Chondroitin sulphate can be obtained from the cartilaginous tissues of animals, such as tracheas of bovine or porcine livestock and cartilaginous skeletons of sharks.

Sodium chondroitin sulphate can be prepared following processes disclosed in the literature (A. D. Nusimovich and F. J. Vila, ES 547769).

The other alkaline and alkaline earth salts can be obtained from sodium chondroitin sulphate by the sodium exchange process by the corresponding cation, following conventional chemical processes.

For use in the treatment or prevention of psoriasis with skin affection, the alkaline or alkaline earth metal chondroitin sulphate of the present invention is formulated in suitable pharmaceutical compositions, using conventional techniques and excipients, such as those described in Remington's Pharmaceutical Science Handbook, Mack Pub. Co., N.Y., USA.

The pharmaceutical compositions of the invention can be administered to the patient in the required dosage. The compositions can be administered in different forms, e.g. oral, intravenous, intraperitoneal, intra-articular, subcutaneous, intramuscular, topical, intradermal or intranasal. The pharmaceutical compositions of the invention include a therapeutically effective quantity of alkaline or alkaline earth metal chondroitin sulphate which comes from an enzymatic hydrolysis of animal cartilage, said quantity depending on many factors, such as the physical condition of the patient, age, sex, specific compound, form of administration and other factors well-known in the art. Furthermore, it will be understood that said dosage of active compound can be administered in single or multiple dose units to provide the desired therapeutic effects. If desired, other therapeutic agents can be used together with those provided by the present invention.

The chondroitin sulphate of the invention is preferably administered to the patient by means of a pharmaceutically acceptable carrier. Said carriers are well known in the art and will generally be in solid or liquid form. Amongst the pharmaceutical preparations in solid form that can be prepared in accordance with the present invention are included powders, mini-granules (pellets), tablets, dispersible granules, capsules, cachets, suppositories and other solid galenical forms. Amongst the liquid preparations are included solutions, suspensions, emulsions, microspheres and nanoparticles. Also included are solid preparations that are to be converted, immediately before being used, in liquid form for oral, parenteral or intra-articular administration. Said liquid forms include solutions, suspensions and emulsions.

An advantage that can be obtained with the invention in comparison with the use of other therapies for the treatment of psoriasis with skin affection, lies in that chondroitin sulphate has no damaging gastric, hepatic and renal effects, and it can be continued to be used for years with no secondary effects.

Another advantage of using chondroitin sulphate that comes from an enzymatic hydrolysis of animal cartilage, lies in the ease of preparation of said chondroitin sulphate from an accessible material such as animal cartilage, following a process which is well-established in the literature. The preparation of an alternative chondroitin sulphate by the in vitro culture of chondrocytes would be economically unfeasible, due to the large quantities of chondrocyte colonies that would be necessary to isolate a small quantity of chondroitin sulphate.

### BRIEF DESCRIPTION OF THE FIGURES

FIG. 1 is an image representative of a longitudinal cut of a skin biopsy corresponding to a psoriatic patient before treatment with chondroitin sulphate (pre-treatment); and

FIG. 2 corresponds to a longitudinal cut of a skin biopsy corresponding to the same patient in FIG. 1 after treatment with chondroitin sulphate (post-treatment). The hematoxylin-eosin stain proves the reduction of the epidermal hyperplasia and vascular tortuousness caused by the treatment with chondroitin sulphate.

### DETAILED DESCRIPTION OF THE INVENTION

The following examples are merely illustrative and do not represent a limitation of the scope of the present invention.

### EXAMPLE 1

### Tablets

The tablets were prepared following conventional processes.

Formula Per Tablet

| | |
|---|---|
| Sodium chondroitin sulphate (13,000-18,000 daltons) | 400.0 mg |
| Avicel PH 200 | 292.0 mg |
| Aerosil 200 | 1.0 mg |
| Magnesium stearate powder | 7.0 mg |

### EXAMPLE 2

### Study in Psoriatic Patients Treated with Chondroitin Sulphate Methodology, Description of the Study

The study was performed on 11 psoriatic patients who were orally administered with 800 mg of chondroitin sulphate daily (two tablets of Example 1, daily), during a two-month period. The parameters determined at the start and end of the treatment were the following:

1.—Clinical evaluation of skin lesions

2.—Histopathological analysis of the skin biopsies

1.—Clinical Evaluation of the Skin Lesions

Basically, the process that occurs in the skin in the case of psoriasis and the effect that was observed after the chondroitin sulphate administration is the following:

A) In psoriatic patients, the skin in the lesions becomes thicker due to the large increase in the number of epidermis cells. The renewal of the external skin cells (epidermis) occurs in 4 days, instead of the usual 25-30 (seven times faster), due to this, the layers of dead skin accumulate, which peel off in the form of scales.

After the administration of chondroitin sulphate, the biopsies examined showed a reduction of hyperkeratosis, i.e. the number of keratinocytes or skin cells that cause the plaques characteristic of psoriasis was reduced. A general improvement in the desquamation of the lesions was observed.

B) In those patients affected with psoriasis, the skins cells do not mature and do not suitably protect, losing humidity due to the lesions.

After the administration of chondroitin sulphate, a spectacular improvement in the hydration of the lesions was observed.

C) The skin of those patients affected with psoriasis has capillaries which are thicker and longer than in normal skin, tangled around one another, and the blood flows in greater quantity. Due to this the plaques appear reddened. The skin is inflamed and the number of immune system cells increases.

After the administration of chondroitin sulphate, some of the patients described a reduction in reddening and the burning sensation which occurs in the lesion.

2.—Histological—anatomopathological Study

The histopathological analysis of 22 skin biopsies corresponding to 11 psoriatic patients before and after treatment with chondroitin sulphate was performed.

Parameters Evaluated:

Quantitative Variables

The total thickness of the epidermis, the maximum thickness from the basal layer to the start of the corneal layer and the maximum thickness of the corneal layer were measured. The number of cells in the proliferation cycle were also determined.

The pre and post-treatment data for each patient were compared, establishing, for each variable, a number of the percentage of reduction or increase in thickness after administering the treatment.

Semi-Quantitative Variables

The degree of activity of the psoriasis was determined, based on its diagnostic criteria (epidermal hyperplasia or acanthosis, parakeratosis, neutrophilic exocytosis and tortuousness of the capillaries of the papillary dermis). Four different degrees (0, 1, 2 and 3) were established and the pre and post-treatment differences were evaluated.

Qualitative Variables

The presence of orthokeratosis (normal keratinization of the skin) or parakeratosis (abnormal keratinization of the skin) was determined in the skin biopsies.

Statistical Analysis

A statistical analysis of the results obtained by an analysis of variance (ANOVA) was made, considering the differences that showed a p less than 0.05 to be significant.

Results:

Thickness of the Epidermis

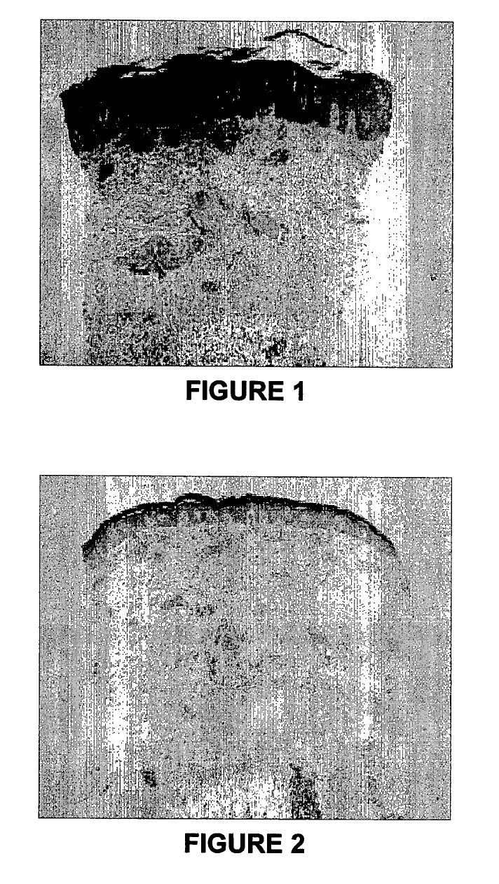

In the three variable studied (total epidermal thickness, maximum thickness from the basal layer to the start of the corneal layer and maximum thickness of the corneal layer), an evident reduction in thickness was observed in the majority of the patients, the maximum percentage of reduction being 61, 69 and 62%, respectively (see Tables 1, 2 and 3). The average reduction was greater in the total epidermal thickness and in the thickness from the basal layer to the start of the corneal layer (30%) than in the thickness of the corneal layer (15%). Therefore, the increase in epidermal thickness shown by the psoriatic patients due to a larger number of keratinocytes or epidermis cells, was notably reduced by treatment with chondroitin sulphate (see FIGS. 1 and 2).

TABLE 1
Maximum epidermal thickness (*p < 0.05)

| | Pre-treatment | Post-treatment | % |
|---|---|---|---|
| Patient | (μm) | (μm) | Difference |
| 1 | 440 | 200 | −55% |
| 2 | 480 | 220 | −54% |
| 3 | 396 | 156 | −61% |
| 4 | 300 | 192 | −36% |
| 5 | 300 | 164 | −45% |
| 6 | 496 | 440 | −11% |
| 7 | 324 | 320 | −1% |
| 8 | 316 | 352 | −11% |
| 9 | 440 | 268 | −39% |
| 10 | 496 | 400 | −19% |
| 11 | 372 | 356 | −4% |

| | |
|---|---|
| % AVERAGE REDUCTION | −29%* |

TABLE 2
Maximum basal-corneal thickness (*p < 0.05)

| | Pre-treatment | Post-treatment | % |
|---|---|---|---|
| Patient | (μm) | (μm) | Difference |
| 1 | 400 | 144 | −64% |
| 2 | 360 | 180 | −50% |
| 3 | 340 | 104 | −69% |
| 4 | 264 | 152 | −42% |
| 5 | 260 | 128 | −51% |
| 6 | 392 | 344 | −12% |
| 7 | 196 | 200 | −2% |
| 8 | 200 | 216 | −8% |
| 9 | 340 | 204 | −40% |
| 10 | 336 | 300 | −11% |
| 11 | 332 | 288 | −13% |

| | |
|---|---|
| % AVERAGE REDUCTION | −31%* |

TABLE 3
Maximum corneal thickness (*p < 0.05)

| | Pre-treatment | Post-treatment | % |
|---|---|---|---|
| Patient | (μm) | (μm) | Difference |
| 1 | 60 | 52 | −13% |
| 2 | 156 | 60 | −62% |
| 3 | 64 | 40 | −38% |
| 4 | 32 | 28 | −13% |
| 5 | 100 | 40 | −60% |
| 6 | 100 | 84 | −16% |
| 7 | 68 | 132 | +94% |
| 8 | 124 | 108 | −10% |
| 9 | 140 | 70 | −50% |
| 10 | 160 | 160 | 0 |
| 11 | 82 | 84 | −2% |

| | |
|---|---|
| % AVERAGE REDUCTION | −15%* |

The measurement of the total epidermal thickness varied between 300 μm and 496 μm before treatment (pre) and between 156 μm and 400 μm thereafter (post) (see Table 1). The maximum basal-corneal thickness varied between 196 μm and 400 μm (pre) and between 104 μm and 344 μm (post) (see Table 2). The maximum thickness of the corneal layer varied between 32 μm and 160 μm (pre) and between 28 μm and 160 μm (post) (see Table 3).

Cellular Proliferation Index

As is observed in Table 4, the results revealed that treatment with chondroitin sulphate reduced the number of cells (keratinocytes) in the proliferation cycle, the average value being a reduction of 28%. Therefore, the high number of dividing keratinocytes which characterize the psoriatic plaques was reduced with the administration of chondroitin sulphate.

TABLE 4
Proliferation index (*p < 0.05)

| Patient | Pre-treatment | Post-treatment | % Difference |
|---|---|---|---|
| 1 | 36 | 11 | −69% |
| 2 | 30 | 24 | −20% |
| 3 | 23 | 6 | −74% |
| 4 | 29 | 21 | −28% |
| 5 | 17 | — | — |
| 6 | 33 | 26 | −21% |
| 7 | 37 | 20 | −46% |
| 8 | 38 | 35 | −8% |
| 9 | 45 | 36 | −20% |
| 10 | 33 | 32 | −3% |
| 11 | 35 | 40 | −14% |

| | |
|---|---|
| % AVERAGE REDUCTION | −28%* |

Degree of Activity of the Psoriasis

Table 5 details the differences found in the degree of activity of the psoriasis, which were also very notable.

TABLE 5
Degree of psoriasis (*p < 0.05)

| Patient | Pre-treatment | Post-treatment | Difference |
|---|---|---|---|
| 1 | 2 | 0 | −2 |
| 2 | 2 | 0.5 | −1.5 |
| 3 | 2 | 0 | −2 |
| 4 | 1 | 1 | 0 |
| 5 | 2 | 1 | −1 |
| 6 | 3 | 2 | −1 |
| 7 | 1 | 1 | 0 |
| 8 | 2 | 2 | 0 |
| 9 | 2 | 1 | −1 |
| 10 | 2 | 1.5 | −0.5 |
| 11 | 0.5 | 0 | −0.5 |

| | |
|---|---|
| AVERAGE REDUCTION | −0.7* |

Presence of Orthokeratosis or Parakeratosis

At the start of the treatment 9 patients showed extensive parakeratosis (PK, abnormal keratinization of the skin) and 2 patients showed a predominant orthokeratotic keratinization (OK, normal keratinization of the skin) with focal areas of PK. On concluding the treatment with chondroitin sulphate, in 6 of those 9 cases described with initial PK, predominant OK with focal PK was observed (see Table 6).

TABLE 6
Orthokeratosis (OK)/Parakeratosis (PK)

| Patient | Pre-treatment | Post-treatment | Difference |
|---|---|---|---|
| 1 | PK | OK | Yes |
| 2 | PK | OK | Yes |
| 3 | PK | OK | Yes |
| 4 | PK | OK | Yes |
| 5 | PK | PK | No |
| 6 | PK | PK | No |
| 7 | OK | OK | No |
| 8 | PK | PK | No |
| 9 | PK | OK | Yes |
| 10 | PK | OK | Yes |
| 11 | OK | OK | No |

### CONCLUSIONS

The 11 psoriatic patients treated with chondroitin sulphate (800 mg/day) for 2 months showed a spectacular clinical improvement of their skin lesions in 100% of the cases. The results of the histopathological study of the biopsies revealed an improvement in the different parameters determined in 70% of the patients. Specifically, a reduction in epidermal thickness was observed, as well as a reduction in the cellular proliferation index of the keratinocytes or skin cells. The degree of activity of the psoriasis also reduced. It is important to emphasize that, histologically, the remaining 30% of the patients did not worsen with the treatment with chondroitin sulphate.

Said effects of chondroitin sulphate observed in psoriatic patients are corroborated with the patients' own evaluation, which was also very positive and satisfactory.

Therefore, it can be concluded that, in patients affected by psoriasis, the oral administration of 800 mg/day of chondroitin sulphate considerably improves the skin lesions caused by said disease, with an excellent safety profile.

1-9. (canceled)

10. A method of treatment or prevention of psoriasis with skin affection in a mammal, comprising administering to a mammal in need thereof an effective amount of an alkaline or alkaline earth metal chondroitin sulfate obtained from enzymatic hydrolysis of animal cartilage.

11. The method of claim 10, wherein said animal cartilage is selected from the group consisting of bovine, porcine and cartilaginous fish cartilage.

12. The method of claim 10, wherein said alkaline metal chondroitin sulfate is sodium chondroitin sulfate.

13. The method of claim 12, wherein said sodium chondroitin sulfate has an average molecular weight between 10,000 and 40,000 daltons.

14. The method of claim 12, wherein said sodium chondroitin sulfate has an average molecular weight between 10,000 and 20,000 daltons.

15. The method of claim 14, wherein said sodium chondroitin sulfate has a sulfur content of 5% to 7% weight/weight, on anhydrous base.

16. The method of claim 10, wherein said administering is oral administering.

17. The method of claim 10, wherein said administering is topical administering.

18. The method of claim 16, wherein said effective amount is from 200 and 3,000 mg of sodium chondroitin sulfate per day.
